(19) United States
(12) Patent Application Publication
(10) Pub. No.: US 2006/0206004 A1
(43) Pub. Date: Sep. 14, 2006

(54) Ureteroscope having a stem

(75) Inventors: Joachim Dehmel, Hamburg (DE); Graham Watson, East Sussex (GB)

Correspondence Address: RANKIN, HILL, PORTER & CLARK LLP, 4080 ERIE STREET, WILLOUGHBY, OH, 44094-7836, US

(73) Assignee: OLYMPUS WINTER & IBE GMBH, Hamburg (DE)

(21) Appl. No.: 11/220,161

(22) Filed: Sep. 6, 2005

(30) Foreign Application Priority Data
Sep. 6, 2004 (DE) 102004043460.3-35

(51) Int. Cl. A61B 1/307

(52) U.S. Cl. 600/128; 600/135

(57) ABSTRACT

A ureteroscope comprising a stem receiving an optics, a light guide and a duct is characterized in that the stem comprises at least along one segment of its length at least one substantially longitudinal indentation.

### BACKGROUND OF THE INVENTION

1. Field of the Invention

The present invention generally relates to and, more particularly, toward ureteroscope stems.

2. Description of Related Art

Ureteroscopes are pushed through the human bladder as far as into the urethra, the connection between bladder and kidney, to allow surgery therein. For that purpose the stem of the ureteroscope must be about 300 to 450 mm long and, due to the urethra's narrowness, must not be significantly larger than 4 mm in diameter.

At least one optics and one light guide, typically a fiber optics bundle, must be received in that very long and thin stem to allow viewing during surgery. Moreover, at least one duct must be provided in order to deploy the urethra in the field of view and, also, to flush the field of view clear in case of bleeding. Moreover, the duct must also be used as an operational conduit to insert surgical implements themselves comprising long stems, for instance blades or forceps, a separate operational duct being precluded by the stem's small cross-section.

As regards two duct ureteroscopes such as described in the applicant's brochure “Two Channel OES 4000 Ureteroscopes” 2003, the two ducts may be used for purposes of forward and return flows. However, the two ducts are perforce narrow and flow-hampering while restricting markedly the available implement cross-section.

As regards ureteroscopes of the above species employing only one duct, this duct does offer advantageous cross-sectional features, but it must be exclusively used to either supply the liquid or to drain it. Accordingly, the procedure may be carried out only intermittently. Where lengthy surgery is concerned, the liquid that was applied to keep the field of view clear subsequently must be drained in order to prevent exceedingly painful renal swelling. Continuous flushing is impossible.

### SUMMARY OF THE INVENTION

Therefore, an objective of the present invention is to create a liquid return duct for ureteroscopes of the above species while preserving an ample cross-section for the insertion of implements.

In accordance with the present invention, the stem is fitted with a longitudinal indentation. The urethra tightly stretched by the ureteroscope stem spans the indentation's cross-section while being prevented from entering it. As a result, a duct is formed at the outside the stem and runs along the stem and may be used as the return duct. Additional indentations moreover may be used which by their total cross-section constitute a larger return duct. Consequently, the design of the invention when applied to a narrow stem comprising solely one liquid supply duct creates an external liquid return duct. This design therefore offers the opportunity to flush continuously.

Known ureteroscopes comprise a stem which is oval in a distal segment and round in a proximal segment, the longer diameter of the oval cross-section corresponding to the round segment diameter. In this manner the stem is optimized in its distal segment for resistance to bending and for the most narrow possible cross-section. The present invention takes advantage of this design. As a result, a cross-section is attained in the middle segment to match in one part the round cross-section of the proximal segment and in another part the oval cross-section of the distal segment. Such design allows easy manufacture and offers high strength on account of the partly unhampered transitions between the segments. At the middle segment, the cross-section merges at two sites from the larger, round zone into the narrower oval zone, forming indentations at these places, the indentations allowing relatively sharp edges at their bottoms and thus allowing being kept clear in each outer duct spanned by the overhead urethra. In this manner very rigid construction is attained having two outer ducts that may be used as return conduits.

### BRIEF DESCRIPTION OF THE DRAWINGS

These and further features of the present invention will be apparent with reference to the following description and drawings, wherein:

FIG. 1 is a sideview of a stem of the ureteroscope of the present invention,

FIGS. 2-4 are sections along lines 2-2 through 4-4 of FIG. 1.

### DETAILED DESCRIPTION OF THE PREFERRED EMBODIMENTS

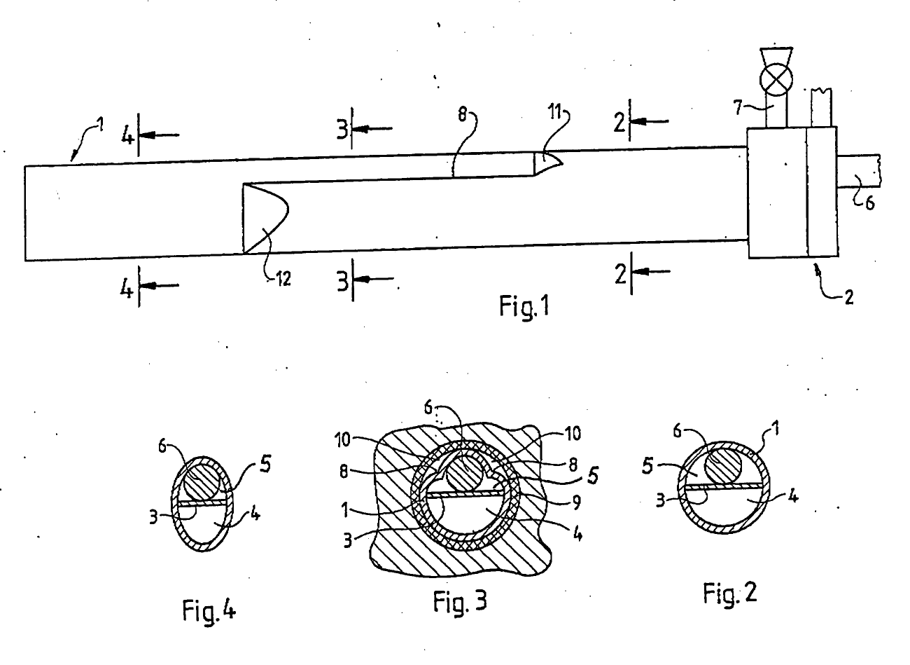

FIG. 1 shows a stem 1 of a ureteroscope which is otherwise omitted, the stem being affixed at its proximal end to a coupling element 2 whereby it may be connected by the omitted handling element of the ureteroscope. This stem is about 450 mm long and its diameter in the plane of the drawing is about 4 mm; for the sake of graphic clarity, this stem is represented in much shortened form.

From one end to the other, the stem 1 is divided at its inside by a longitudinally sub-dividing wall 3 into a duct 4 and a residual space 5 receiving an optics 6, and further, in the remaining zone of its cross-section, a light guide, such as a fiber optic bundle for transmitting light to the space to be observed. The longitudinally sub-dividing wall 3 moreover may be eliminated (not shown) in the manner of conventional designs. The optics 6 may be retractable and may receive the light guide in parts of its own cross-section.

The duct 4 is connected to a supply hookup element 7 through which it receives flushing liquid that issues at the distal end of the stem 1 and enlarges the field of view of the optics 6 and flushes out interfering impurities, such as blood. Furthermore, an omitted surgical implement of long shank—for instance a blade or forceps—may be inserted through the supply hookup element 7 into the duct 4, in which case the supply hookup element shall not be perpendicular to the stem 1 in the manner shown in FIG. 1 but instead shall be oblique to it.

As shown by FIG. 2, the stem of FIG. 1 is cross-sectionally round in a proximal segment of its length. As shown by FIG. 4, the distal cross-section of the stem 1 is oval.

In a middle segment between the proximal and the distal segments, the cross-section of stem 1 is as shown in FIG. 3. The cross-section of the upper zone of FIG. 3 corresponds substantially to that of FIG. 4 and the cross-section of FIG. 3 in its lower zone corresponds to that of FIG. 2. Indentations 8 are formed in each case in the transition range between the two cross-section parts, as shown in FIG. 3.

FIG. 3 shows the stem 1 within a urethra 9. This urethra is much enlarged by the cross-section of the stem 1 and is stressed against the periphery of this stem 1. Accordingly, the urethra is taut while spanning the indentations 8 and thus cannot enter them. In this manner an outer duct 10 is subtended at each indentation 8.

When liquid flows constantly into and through the inner duct 4 in the stem 1, this liquid then may flow back externally through the zone of oval cross-section (FIG. 4) because the overall cross-section there is less and the urethra 9 does enclose less tightly. In the middle segment of the stem 1 (FIG. 3), the liquid is able to flow back through the outer ducts 10. In the proximal segment (FIG. 2), the stem 1 lies freely in the bladder and an external duct no longer is needed.

The cross-section of FIG. 3 merges in gradual and non-traumatic manner in the transition ranges 11, 12 into the cross-sections of FIGS. 2 and 4, respectively.

In variations of the shown embodiment, the stem 1 might be round everywhere or it might abruptly change from a round into an oval cross-section. Those segments requiring tight enclosure by the urethra may be fitted with longitudinal indentations, other than those shown in FIGS. 1 and 3 at the stem periphery, such indentations then exhibiting a cross-sectional U or V shape and offering adequate duct cross-section by means of their indentation cross-section, though, if feasible, they also may be configured in less interfering manner in the inside space.

In the shown embodiment the indentations 8 run parallel to the stem axis, that is in the longitudinal direction. Alternatively however and while retaining their overall longitudinal direction, they also may be configured in oblique manner, for instance helically or sinusoidally.

1. A ureteroscope having a stem (1) receiving an optics (6), a light guide and a duct (4),
characterized in that
the stem (1) comprises in at least one segment of its length at least one indentation (8) running in its longitudinal direction.

2. Ureteroscope as claimed in claim 1, comprising a proximal segment of the stem (1) exhibiting a round cross-section and a distal segment exhibiting an oval cross-section, characterized in that, in a middle segment of the stem (1), the cross-section (FIG. 3) corresponds in one peripheral portion to the round cross-section (FIG. 2) and in another peripheral portion it corresponds substantially to the oval cross-section (FIG. 4).
